(19) United States
(12) Patent Application Publication
(10) Pub. No.: US 2004/0097432 A1
(43) Pub. Date: May 20, 2004

(54) Method of reducing cholesterol

(75) Inventors: Haeri Roh-Schmidt, Ada, MI (US); James B. Roufs, Santa Monica, CA (US)

Correspondence Address: G. PETER NICHOLS, Brinks Hofer Gilson & Lione, NBC Tower, Suite 3600, P.O. Box 10395, Chicago, IL, 60610, US

(73) Assignee: ACCESS BUSINESS GROUP INTERNATIONAL LLC.

(21) Appl. No.: 10/700,222

(22) Filed: Nov. 3, 2003

Related U.S. Application Data
(60) Provisional application No. 60/423,612, filed on Nov. 4, 2002.

(51) Int. Cl. A61K 31/7048; A61K 31/353; A61K 35/78

(52) U.S. Cl. 514/27; 514/456; 424/729

(57) ABSTRACT

A method of treating elevated cholesterol levels, hyperlipidemia and/or hypercholesterolemia in a mammal includes administering an effective amount of theaflavins, thearubigins, and their mixture. The theaflavins include theaflavin and gallate esters of theaflavin, particularly those obtained from tea. The gallate esters include theaflavin-3-gallate, theaflavin-3′-gallate, and theaflavin-3,3′-digallate.

The present invention claims priority to U.S. Ser. No. 60/423,612 filed Nov. 4, 2002, the entire contents of which are incorporated herein by reference.

The present invention relates to compositions containing theaflavins, thearubigins, or their combination for reducing cholesterol and for the treatment of hyperlipidemia and/or hypercholesterolemia. In particular, the present invention is directed to the method of reducing cholesterol or treating hyperlipidemia and/or hypercholesterolemia in a mammal by administering an anti-hyperlipidemia and/or anti-hypercholesterolemia effective amount of theaflavins, thearubigin, or their mixture. A desired composition includes a neutriceutically acceptable diluent or carrier and an active ingredient that is selected from the group consisting of theaflavin, a gallate ester of theaflavin, or their mixture, wherein the theaflavin and gallate ester of theaflavin are derived from tea.

There is ongoing interest in reducing, treating or regulating cholesterol levels in the body because of the known link between hyperlipidemia and hypercholesterolamia and cardiovascular disease. A popular drug, Lipitor®, is prescribed to lower the lipid content in hyperlipidemic people. Despite the success of Lipitor®, many people desire a natural alternative to the widely available prescription drugs.

In this regard, it has been suggested that the ingestion of tea may be beneficial in treating or preventing cardiovascular disease. Green tea leaf (as picked) contains colorless polyphenols known as catechins. The four major catechins in green tea leaf are epicatechin (EC) and epigallocatechin (EGC) and the gallated forms of these catechins (bearing a gallic acid (GA) residue), epicatechin-3-gallate (ECG) and epigallocatechin-3-gallate (EGCG). It is believed that these catechins are responsible for lowering blood cholesterol and decreasing lipoprotein of low density, although the clinical data demonstrates minor effects.

During oxidative fermentation of green leaf to produce black tea (for example by solid state fermentation to produce black leaf or slurry fermentation to produce black tea extracts), the catechins undergo oxidative biotransformations, through their quinones, into dimeric compounds known as theaflavins (TFs) and higher molecular weight compounds known as thearubigins (TRs). TFs and TRs are responsible for the orange and brown colors of black tea infusions and products as well as for the astringency and body of the made tea. TRs are larger in size and darker in color than TFs. The oxidative polymerizations are a combination of biochemical oxidations mediated by polyphenol oxidase and/or peroxidase enzymes present in the leaf and chemical reactions of reactive species. TFs include theaflavin and a range of related gallated derivatives (gallate esters of theaflavin).

It has now been found that the theaflavins derived from tea, and in particular theaflavin and the gallate esters of theaflavin are useful in treating hyperlipidemia and/or hypercholesterolemia. Accordingly, compositions that contain an anti-hyperlipidemia and/or an anti-hypercholesterolemia effective amount of theaflavins selected from the group consisting of theaflavin and the gallate esters of theaflavin are believed to provide a natural alternative to Lipitor® and other prescription drugs.

### SUMMARY OF THE INVENTION

The scope of the present invention is defined solely by the appended claims, and is not affected to any degree by the statements within this summary. By way of introduction, a method of treating hyperlipidemia and/or hypercholesterolemia in a mammal includes administering to the mammal an anti-hyperlipidemia and/or an anti-hypercholesterolemia effective amount of theaflavins, thearubigins, and mixtures thereof. In this regard the present invention contemplates a method of inhibiting cholesterol synthesis and/or reducing cholesterol and thereby treating hyperlipidemia and/or hypercholesterolemia.

In a second aspect of the present invention a neutraceutical composition is provided for the treatment of hyperlipidemia and/or hypercholesterolemia that comprises an anti-hyperlipidemia and/or an anti-hypercholesterolemia effective amount of theaflavins, thearubigins, and mixtures thereof. The composition may further include a neutraceutically acceptable diluent or carrier.

It is believed that the thearubigins may be useful for treating hyperlipidemia and/or hypercholesterolemia. In this regard, it is believed that the thearubigins can be used alone or in combination with the theaflavins for the treatment of hyperlipidemia and/or hypercholesterolemia.

In another aspect of the present invention, a kit is provided for treating hyperlipidemia and/or hypercholesterolemia that contains an anti-hyperlipidemia and/or anti-hypercholseterolemia effective amount of theaflavins, thearubigins, or their mixture. In particular, the theaflavins are selected from theaflavin, gallate esters of theaflavin, or their mixture.

### BRIEF DESCRIPTION OF THE DRAWINGS

FIG. 1 shows the dose specific response of HepG2 cells to theaflavins and Lipotor® in cholesterol release response of hepatocytes.

FIG. 2 shows the dose specific response of HepG2 cells to theaflavin, gallate esters of theaflavin, theaflavins, and catechin in cholesterol release response of hepatocytes.

### DESCRIPTION OF THE INVENTION

It has now been discovered that theaflavins derived from tea are effective in treating hyperlipidemia and/or hypercholesterolemia. Accordingly, in one aspect of the present invention, a method of treating hyperlipidemia and/or hypercholesterolemia in a mammal includes administering to the mammal an anti-hyperlipidemia and/or an anti-hypercholesterolemia effective amount of theaflavins, thearubigins, and mixtures thereof. In this aspect, a composition is provided for use in treating hyperlipidemia and/or hypercholesterolemia and which comprises an effective amount of theaflavins, thearubigins, and mixtures thereof.

The term “theaflavins” collectively describes those compounds that are formed by the enzymatic oxidation and condensation product of tea catechins with di- and trihydroxylated B rings. For example, the term “theaflavins” includes theaflavin and its gallated derivatives, i.e., gallate esters of theaflavin. The term “gallate esters of theaflavin” includes those compounds of formula [I]:

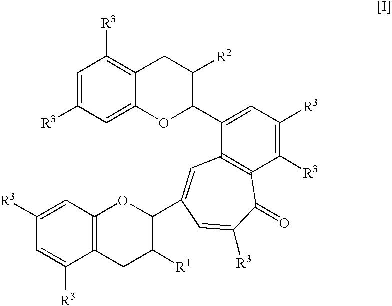

wherein R¹, R², and R³ are each OH or the compound of formula [A]:

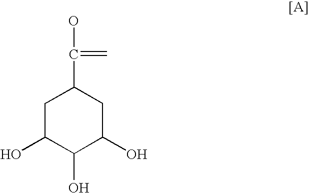

Desired gallate esters of theaflavin includes those compounds of formula [II]:

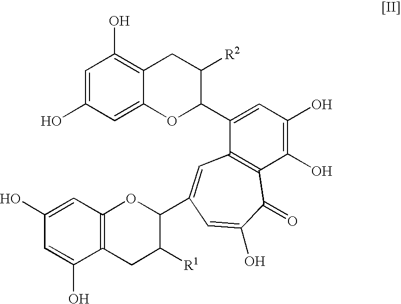

wherein R¹ and R² are each OH or the compound of formula [A].

More desired gallate esters of theaflavin include the compounds of formula [III]:

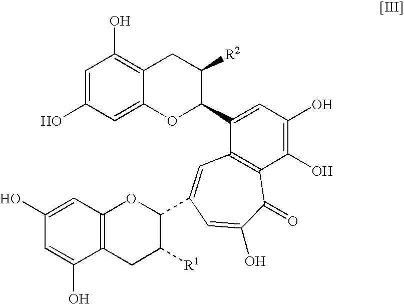

wherein R¹ and R² are each OH or the compound of formula [A]. A more desired gallate ester of theaflavin is theaflavin-3-gallate.

The theaflavins of formula [I] contain multiple asymmetric carbon centers and thus they can exist in a variety of different stereoisomeric forms. For example, the substituents attached at the chiral two- and three-positions of the theaflavin tetrahydropyran rings may have cis- or trans-stereochemistry relative to one another and relative to other substituents attached at other chiral centers elsewhere in the theaflavin. Accordingly, except where specifically noted, the theaflavins of formula [I] include all possible optically pure single stereoisomers as well as all possible mixtures thereof.

A desired method includes providing an anti-hyperlipidemia and/or anti-hypercholesterolemia effective amount of theaflavins. More desirably, the method includes providing an anti-hyperlipidemia and/or anti-hypercholesterolemia effective amount of theaflavins selected from theaflavin, theaflavin-3-gallate, theaflavin-3′-gallate, theaflavin-3,3′-digallate, and mixtures thereof, wherein the theaflavins are derived from tea.

Thearubigins are those compounds that are even more extensively oxidized and polymerized than the theaflavins and they have a wide range of molecular weights and are not very well characterized. A suggested structure is shown below:

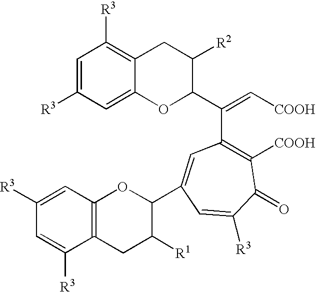

wherein R¹, R², and R³ are each OH or the compound of formula [A].

It is believed that the thearubigins may be effective in treating hyperlipidemia and/or hypercholesterolemia. Accordingly, one aspect of the present invention may include providing an amount of thearubigins effective to treat hyperlipidemia and/or hypercholesterolemia. It is also contemplated that the thearubigins may be used alone or in combination with theaflavins, particularly theaflavin and gallate esters of theaflavin derived from tea.

As noted above, the theaflavins and thearubigins are desirably derived from tea. The term “tea” as used in the present specification and claims is not to be limited to any particular type of tea but refers to material that is usually obtained from tea leaves such as fresh tea leaves, unfermented tea leaves, semi-fermented tea leaves, green teas of medium grade, instant green tea, and black tea. Accordingly, the term “tea” as used in the present specification and claims means material obtained from Camellia sinsensis or Camellia assamica, and blends of these.

It is believed that any suitable manner of obtaining or deriving the theaflavins and/or thearubigins from tea known to those skilled in the art can be used. In this regard, the manner of obtaining and deriving includes isolating the theaflavins and thearubigins by any suitable manner. Theaflavins and/or thearubigins may be commercially available from Nashai and it is believed that the theaflavins and thearubigins are derived from green tea in a manner to control the oxidation of the catechins to theaflavins and to limit the oxidation of the catechins to thearubigins.

It is contemplated that the theaflavins and thearubigins can be administered in any manner suitable to provide an effective amount to a mammal. Accordingly, all manner of oral dosage forms suitable for peroral administration of a pharmaceutical or neutraceutical are contemplated for use in accordance with the present invention. Representative oral dosage forms for use in accordance with the present invention include but are not limited to pills, capsules, gel caps, gel tabs, beverages, chewing gums, chewable tablets, lozenges, viscous gels, troches, toothpastes, dental implants, gargling gels, mouth rinses, and the like, and combinations thereof. Presently preferred oral dosage forms include pills, capsules, gel caps, gel tabs, chewable tablets, lozenges, and troches.

The pharmaceutical or neutraceutical compositions may take the form of, for example, tablets or capsules prepared by conventional means with pharmaceutically or neutraceutically acceptable diluents, carriers, or excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art.

The compositions for oral administration may also be formulated to give controlled release of the active compounds. In this regard, the active compounds of the present invention may be formulated as controlled release powders of discrete micro-particles that can be readily formulated in liquid form. The sustained release powder comprises particles containing an active ingredient and optionally, an excipient with at least one non-toxic polymer.

The powder can be dispersed or suspended in a liquid vehicle and will maintain its sustained release characteristics for a useful period of time. These dispersions or suspensions have both chemical stability and stability in terms of dissolution rate. The powder may contain an excipient comprising a polymer, which may be soluble, insoluble, permeable, impermeable, or biodegradable. The polymers may be polymers or copolymers. The polymer may be a natural or synthetic polymer. Natural polymers include polypeptides (e.g., zein), polysaccharides (e.g., cellulose), and alginic acid.

The compositions of the present invention may, if desired, be presented in a pack or dispenser device that may contain one or more unit dosage forms containing the active ingredients. The pack may for example comprise metal or plastic foil, such as a blister pack. The pack or dispenser device may be accompanied by instructions for administration.

The invention also provides kits for carrying out the therapeutic regimens of the invention. Such kits comprise one or more containers having therapeutically or prophylactically effective amounts of the theaflavins and/or thearubigins in a pharmaceutically or neutraceutically acceptable form. The theaflavins or thearubigins in a vial or a kit of the invention may be in the form of a pharmaceutically or neutraceutically acceptable solution, e.g., in combination with sterile saline, dextrose solution, or buffered solution, or other pharmaceutically or neutraceutically acceptable sterile fluid. Instructions may be printed (e.g., on paper) and/or supplied in an electronic-readable medium (e.g., floppy disc, CD-ROM, DVD-ROM, zip disc, videotape, audio tape, etc.). Alternatively, instructions may be provided by directing a user to an Internet web site (e.g., specified by the manufacturer or distributor of the kit) and/or via electronic mail.

In one aspect of this invention, the compositions according to the present invention may be used as a dietary or nutritional supplement for the treatment of hyperlipidemia and/or hypercholesterolemia. In this aspect, the total daily dose ranges of the active theaflavins and/or thearubigins for the conditions described herein are generally from about 1 mg to about 800 mg administered in divided doses administered parenterally or orally. A preferred total daily dose is from about 25 mg to about 400 mg of the active theaflavins and/or thearubigins.

In another embodiment, a total daily dose of a sustained release formulation may be used as a dietary supplement is about 1 mg to about 800 mg of active theaflavins and/or thearubigins administered twice daily (e.g., in the morning and the evening) at a dose of about 0.5 mg to about 400 mg. The dosage forms and compositions may comprise any of the forms and compositions described above. In a preferred embodiment, the formulation is a tablet, capsule, gel, or a liquid-soluble powder.

The following examples illustrate, but do not limit, the present invention.

### EXAMPLES

### Example I

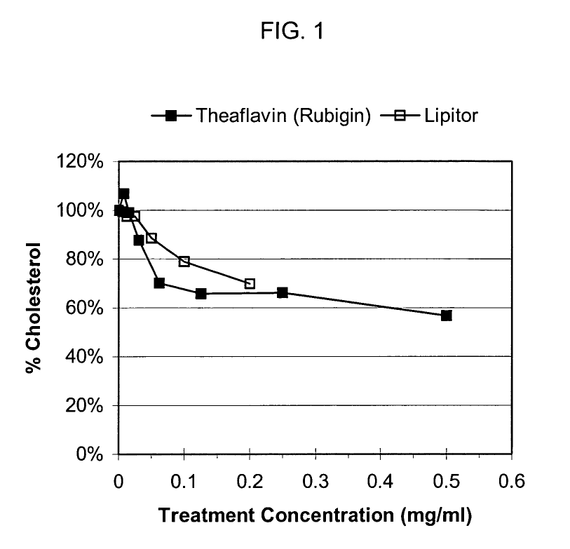

HepG2 hepatocytes were used as a model to study the inhibition of cholesterol synthesis according to the procedures described in Dashti, N., et al., J. Lipid Res. 28:423-436 (1987) and Mohammadi, A., et al., Arterioscler. Thromb. Vasc. Biol., 185:783-793 (1998). Lipotor® was prepared in methanol/buffer to solubilize Atorvastatin, the active ingredient (40 mg active/600 mg tablet) in Lipotor®. Amounts of secreted cholesterol and cholesteryl ester are measured from acetate fed hepatocyte culture media using a fluorescent indicator AmplexRed. FIG. 1 shows the dose specific response of hepatocytes to theaflavin as compared to Lipotor® and it is found to be comparable or slightly better than Lipotor® on a same weight basis. It is noted that the weight used for Lipitor® contains 6.7% actives (Atorvastatin per tablet). Accordingly, it appears that a 30% inhibitory effect is achieved by 0.05 mg/ml theaflavin which relates to 0.2 mg/ml Lipitor® tablet that contains 0.014 mg/ml Atorvastatin.

### Example II

The same cholesterol inhibition model used in Example I was used to determine the cholesterol synthesis inhibition of theaflavins, including theaflavin and gallate esters of theaflavin. Table I shows the materials and amounts tested:

TABLE I

| Material Tested | Actual Dose (ug/ml) |
|---|---|
| Theaflavin (T) | 4, 2, 1, 0.5, 0.25 and 0 |
| Theaflavin monogallate (TG) | 13, 6.5, 3.25, 1.625, 0.8125 and 0 |
| Theaflavin digallate (TGG) | 26, 13, 6.5, 3.25, 1.625 and 0 |
| Epigallocatechin gallate (EGCG) | 50, 25, 12.5, 6.25, 3.125 and 0 |
| Catechin (C) | 4, 2,1, 0.5, 0.25 and 0 |
| Green Tea Extract (CD1705) | 200, 100, 50, 25, 12.5 and 0 |

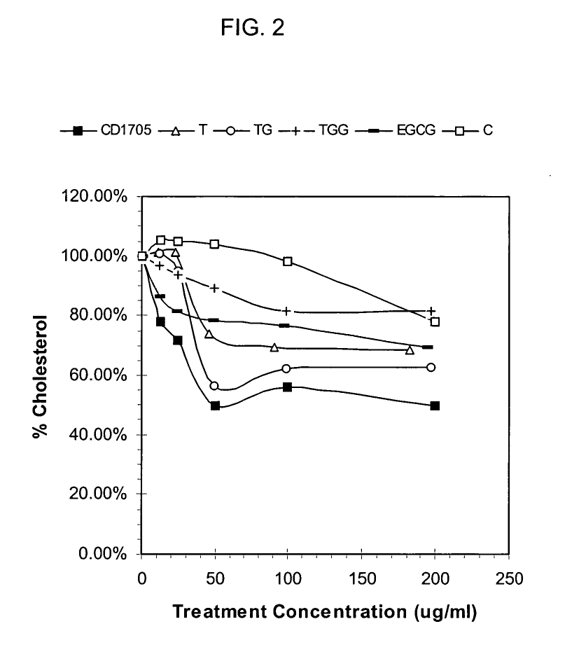

The green tea extract contained 22% theaflavins of which 60% was theaflavin-3,3′-digallate, 30% theaflavin-3-gallate, and 10% theaflavin. The green tea extract also contained 57.3% catechins of which 45% was epigallocatechin gallate, 3.5% catechin, and the balance was undefined. FIG. 2 shows the dose specific response of hepatocytes to the material tested. It was unexpectedly found that the theaflavin-3-gallate provided much of the cholesterol inhibition activity.

The foregoing detailed description and examples have been provided by way of explanation and illustration, and are not intended to limit the scope of the appended claims. Many variations in the presently preferred embodiments illustrated herein will be obvious to one of ordinary skill in the art, and remain within the scope of the appended claims and their equivalents.

What is claimed:

1. A method for reducing cholesterol in a mammal comprising administering an effective amount of theaflavins.

2. The method of claim 1 wherein the theaflavins are selected from the group consisting of theaflavin, a gallate ester of theaflavin, and mixtures thereof.

3. The method of claim 1 wherein the theaflavins are derived from tea.

4. The method of claim 2 wherein the gallate ester of theaflavin is selected from the group consisting of theaflavin monogallate, theaflavin digallate, and mixtures thereof.

5. The method of claim 2 wherein the gallate ester of theaflavin is selected from the group consisting of theaflavin-3-gallate, theaflavin-3′-gallate, theaflavin-3,3′-digallate, and mixtures thereof.

6. The method of claim 5 wherein the gallate ester of theaflavin is theaflavin-3-gallate.

7. The method of claim 6 wherein the theaflavin-3-gallate is derived from tea.

8. The method of claim 1 wherein the amount of theaflavins ranges from about 1 mg to about 800 mg.

9. The method of claim 2 wherein the amount of theaflavins ranges from about 1 mg to about 800 mg.

10. A method for treating hyperlipidemia in a mammal comprising administering an effective amount of an anti-hyperlipidemia compound selected from the group consisting of theaflavin, a gallate ester of theaflavin, and mixtures thereof.

11. The method of claim 10 wherein the anti-hyperlipidemia compound is derived from tea.

12. The method of claim 10 wherein the gallate ester of theaflavin is selected from the group consisting of theaflavin monogallate, theaflavin digallate, and mixtures thereof.

13. The method of claim 10 wherein the gallate ester of theaflavin is selected from the group consisting of theaflavin-3-gallate, theaflavin-3′-gallate, theaflavin-3,3′-digallate, and mixtures thereof.

14. The method of claim 13 wherein the gallate ester of theaflavin is theaflavin-3-gallate.

15. The method of claim 14 wherein the theaflavin-3-gallate is derived from tea.

16. The method of claim 10 wherein the amount of the anti-hyperlipidemia compound ranges from about 1 mg to about 800 mg.
